(19) United States
(12) Patent Application Publication
(10) Pub. No.: US 2020/0155510 A1
(43) Pub. Date: May 21, 2020

(54) TREATMENT OF AUTOIMMUNE DISORDERS, SUCH AS RELAPSING REMITTING MULTIPLE SCLEROSIS AND CLINICALLY ISOLATED SYNDROME WITH BIOTIN COMPOSITIONS

(71) Applicant: JDS Therapeutics, LLC, Purchase, NY (US)

(72) Inventor: James R. Komorowski, Trumbull, CT (US)

(21) Appl. No.: 16/681,285

(22) Filed: Nov. 12, 2019

Related U.S. Application Data
(60) Provisional application No. 62/760,581, filed on Nov. 13, 2018.

(51) Int. Cl. A61K 31/4188

(52) U.S. Cl. CPC A61K 31/4188

(57) ABSTRACT

The present application relates to the treatment of autoimmune disorders, such as CIS or RRMS using biotin compositions. The present application also relates to the delay of progression from CIS to RRMS and RRMS to PPMS using biotin compositions. Also disclosed are methods of reducing pro-inflammatory cytokines using biotin compositions.

### CROSS-REFERENCE TO RELATED APPLICATIONS

This application claims priority to of U.S. Provisional Application No. 62/760,581 filed Nov. 13, 2018, the contents of which is incorporated by reference herein in its entirety.

### BACKGROUND

### Field

The present application relates to the treatment of autoimmune disorders, such as relapsing remitting multiple sclerosis and clinically isolated syndrome, reduction of pro-inflammatory cytokines and delay of progression to primary progressive multiple sclerosis with biotin compositions.

### Description of the Related Art

Biotin is an essential water-soluble vitamin also known as Vitamin H, Coenzyme R, and Vitamin B7. It is an essential co-factor for five known carboxylases involved in fatty acid biosynthesis, gluconeogenesis, branched-chain amino acid metabolism, fatty acid metabolism, tricarboxylic acid cycle anaplerosis, and pleiotropic gene regulation, particularly for genes in carbohydrate metabolism. Magnesium biotinate is more water soluble than biotin.

Relapsing remitting multiple sclerosis (“RRMS”) is an autoimmune disease and involves inflammatory attacks on myelin, as well as the nerve fibers themselves. During these inflammatory attacks, activated immune cells cause small, localized areas of damage. RRMS is different than primary progressive multiple sclerosis (“PPMS”) in that RRMS involves more brain lesions and these lesions contain more inflammatory cells, whereas PPMS involves more spinal cord lesions, which contain fewer inflammatory cells. RRMS is usually diagnosed in people in their twenties and thirties, whereas PPMS is usually diagnosed in people in their forties and fifties. People with RRMS for ten or more years usually transition to PPMS. Common symptoms reported in RRMS include episodic bouts of fatigue, numbness, vision problems, spasticity or stiffness, bowel and bladder problems, and problems with cognition. Common symptoms reported in PPMS are gradually worsening problems with walking and mobility. Inflammatory markers, including IL-17 and TNF-α are associated with RRMS.

Clinically isolated syndrome (“CIS”) is an autoimmune disease and refers to a first episode of neurologic symptoms that lasts at least 24 hours and is caused by inflammation or demyelination in the central nervous system. CIS can be monofocal, or multifocal. Monofocal refers to a single neurologic sign or symptom, for example, an attack of optic neuritis, which is caused by a single lesion. Multifocal refers to more than one sign or symptom, for example, an attack of optic neuritis accompanied by numbness or tingling in the legs, which is caused by lesions in more than one place. CIS differs from PPMS because CIS is the first episode of inflammation or demyelination in the central nervous system, PPMS is more than one, and usually many episodes of inflammation or demyelination in the central nervous system. Individuals who experience CIS may or may not go on to develop PPMS. When CIS is accompanied by magnetic resonance imaging (MRI)-detected brain lesions that are similar to those seen in PPMS, the person has a 60 to 80 percent chance of a second neurologic event and diagnosis of PPMS within several years. When CIS is not accompanied by MM-detected brain lesions, the person has about a 20 percent chance of developing PPMS over the same period of time.

### SUMMARY OF THE INVENTION

In some embodiments, biotin will be used to treat an autoimmune disorder of a human's central nervous system. In some embodiments, a biotin salt will be used to treat an autoimmune disorder of a human's central nervous system. In some embodiments magnesium biotinate will be used to treat an autoimmune disorder of a human's central nervous system. In some embodiments, the type of autoimmune disorder of a human's central nervous system to be treated may differ. In some embodiments, the type of autoimmune disorder of a human's central nervous system to be treated may be CIS, RRMS or another autoimmune disorder of a human's central nervous system in which inflammation and/or demyelination are involved. In some embodiments, the type of autoimmune disorder of a human's central nervous system to be treated may involve elevated levels of pro-inflammatory cytokines. In some embodiments, the amount of biotin, biotin salt or magnesium biotinate administered is between 10 mg/day to 10000 mg/day. In some embodiments, the amount of biotin, biotin salt or magnesium biotinate administered is between 100 mg/day and 1000 mg/day. In some embodiments, the amount of biotin, biotin salt or magnesium biotinate administered is between 30 mg/day and 300 mg/day. In some embodiments, the biotin, biotin salt, or magnesium biotinate will be administered once a day. In some embodiments, the biotin, biotin salt, or magnesium biotinate will be administered more than once a day. In some embodiments, the biotin, biotin salt, or magnesium biotinate will be administered via an oral route. In some embodiments, the biotin, biotin salt, or magnesium biotinate will be administered via an intraperitoneal route. In some embodiments, the biotin, biotin salt, or magnesium biotinate will be administered via a transdermal, rectal, or sublingual route. In some embodiments, the amount of magnesium biotinate administered is between 10 mg/day to 1000 mg/day. In some embodiments, the amount of magnesium biotinate administered is between 30 mg/day and 300 mg/day. In some embodiments, the amount of magnesium biotinate administered is equivalent to between 10 mg/day of biotin to 1000 mg/day of biotin. In some embodiments, the biotin, biotin salt, or magnesium biotinate can be provided as a drug, supplement, medical food, food or biologic. In some embodiments, the biotin, biotin salt, or magnesium biotinate is administered alone. In some embodiments, the biotin, biotin salt, or magnesium biotinate is administered in combination with another treatment. In some embodiments, the biotin, biotin salt, or magnesium biotinate is administered for 1 or more days. In some embodiments, the biotin, biotin salt, or magnesium biotinate is administered for 1 or more weeks. In some embodiments, the biotin, biotin salt, or magnesium biotinate is administered for 1 or more months. In some embodiments, the biotin, biotin salt, or magnesium biotinate is administered for 1 or more years.

In some embodiments, the invention provides a method of delaying progression from CIS to RRMS comprising administering an effective amount of biotin, biotin salt, or magnesium biotinate. In some embodiments, the amount of biotin, biotin salt or magnesium biotinate administered is between 10 mg/day to 10000 mg/day. In some embodiments, the amount of biotin, biotin salt or magnesium biotinate administered is between 100 mg/day and 1000 mg/day. In some embodiments, the amount of biotin, biotin salt or magnesium biotinate administered is between 30 mg/day and 300 mg/day. In some embodiments, the biotin, biotin salt, or magnesium biotinate will be administered once a day. In some embodiments, the biotin, biotin salt, or magnesium biotinate will be administered more than once a day. In some embodiments, the biotin, biotin salt, or magnesium biotinate will be administered via an oral route. In some embodiments, the biotin, biotin salt, or magnesium biotinate will be administered via an intraperitoneal route. In some embodiments, the biotin, biotin salt, or magnesium biotinate will be administered via a transdermal, rectal, or sublingual route. In some embodiments, the amount of magnesium biotinate administered is between 10 mg/day to 1000 mg/day. In some embodiments, the amount of magnesium biotinate administered is between 10 mg/day and 100 mg/day. In some embodiments, the amount of magnesium biotinate administered is between 30 mg/day and 300 mg/day. In some embodiments, the amount of magnesium biotinate administered is equivalent to between 10 mg/day of biotin to 1000 mg/day of biotin. In some embodiments, the biotin, biotin salt, or magnesium biotinate can be provided as a drug, supplement, medical food, food or biologic. In some embodiments, the biotin, biotin salt, or magnesium biotinate is administered alone. In some embodiments, the biotin, biotin salt, or magnesium biotinate is administered in combination with another treatment. In some embodiments, the biotin, biotin salt, or magnesium biotinate is administered for 1 or more days. In some embodiments, the biotin, biotin salt, or magnesium biotinate is administered for 1 or more weeks. In some embodiments, the biotin, biotin salt, or magnesium biotinate is administered for 1 or more months. In some embodiments, the biotin, biotin salt, or magnesium biotinate is administered for 1 or more years.

In some embodiments, the invention provides a method of delaying progression from RRMS to PPMS comprising administering an effective amount of biotin, biotin salt, or magnesium biotinate. In some embodiments, the amount of biotin, biotin salt or magnesium biotinate administered is between 10 mg/day to 10000 mg/day. In some embodiments, the amount of biotin, biotin salt or magnesium biotinate administered is between 100 mg/day and 1000 mg/day. In some embodiments, the amount of biotin, biotin salt or magnesium biotinate administered is between 30 mg/day and 300 mg/day. In some embodiments, the biotin, biotin salt, or magnesium biotinate will be administered once a day. In some embodiments, the biotin, biotin salt, or magnesium biotinate will be administered more than once a day. In some embodiments, the biotin, biotin salt, or magnesium biotinate will be administered via an oral route. In some embodiments, the biotin, biotin salt, or magnesium biotinate will be administered via an intraperitoneal route. In some embodiments, the biotin, biotin salt, or magnesium biotinate will be administered via a transdermal, rectal, or sublingual route. In some embodiments, the amount of magnesium biotinate administered is between 10 mg/day to 1000 mg/day. In some embodiments, the amount of magnesium biotinate administered is between 10 mg/day and 100 mg/day. In some embodiments, the amount of magnesium biotinate administered is between 30 mg/day and 300 mg/day. In some embodiments, the amount of magnesium biotinate administered is equivalent to between 10 mg/day of biotin to 1000 mg/day of biotin. In some embodiments, the biotin, biotin salt, or magnesium biotinate can be provided as a drug, supplement, medical food, food or biologic. In some embodiments, the biotin, biotin salt, or magnesium biotinate is administered alone. In some embodiments, the biotin, biotin salt, or magnesium biotinate is administered in combination with another treatment. In some embodiments, the biotin, biotin salt, or magnesium biotinate is administered for 1 or more days. In some embodiments, the biotin, biotin salt, or magnesium biotinate is administered for 1 or more weeks. In some embodiments, the biotin, biotin salt, or magnesium biotinate is administered for 1 or more months. In some embodiments, the biotin, biotin salt, or magnesium biotinate is administered for 1 or more years.

In some embodiments, the invention provides a method of reducing a level of one or more pro-inflammatory cytokines in a human comprising administering an effective amount of biotin, biotin salt, or magnesium biotinate, wherein the level of one or more pro-inflammatory cytokines is reduced. In some embodiments, the one or more pro-inflammatory cytokines are selected from the group consisting of tumor necrosis factor alpha (TNF-α), interleukin 6 (IL-6), interleukin 17 (IL-17), chemokine (C-C motif) ligand 3 (CCL-3), chemokine (C-C motif) ligand 5 (CCL-5) and chemokine (C-X-C motif) ligand 16 (CXCL-16). In some embodiments, the amount of biotin, biotin salt or magnesium biotinate administered is between 10 mg/day to 10000 mg/day. In some embodiments, the amount of biotin, biotin salt or magnesium biotinate administered is between 100 mg/day and 1000 mg/day. In some embodiments, the amount of biotin, biotin salt or magnesium biotinate administered is between 30 mg/day and 300 mg/day. In some embodiments, the biotin, biotin salt, or magnesium biotinate will be administered once a day. In some embodiments, the biotin, biotin salt, or magnesium biotinate will be administered more than once a day. In some embodiments, the biotin, biotin salt, or magnesium biotinate will be administered via an oral route. In some embodiments, the biotin, biotin salt, or magnesium biotinate will be administered via an intraperitoneal route. In some embodiments, the biotin, biotin salt, or magnesium biotinate will be administered via a transdermal, rectal, or sublingual route. In some embodiments, the amount of magnesium biotinate administered is between 10 mg/day to 1000 mg/day. In some embodiments, the amount of magnesium biotinate administered is between 10 mg/day and 100 mg/day. In some embodiments, the amount of magnesium biotinate administered is between 30 mg/day and 300 mg/day. In some embodiments, the amount of magnesium biotinate administered is equivalent to between 10 mg/day of biotin to 1000 mg/day of biotin. In some embodiments, the biotin, biotin salt, or magnesium biotinate can be provided as a drug, supplement, medical food, food or biologic. In some embodiments, the biotin, biotin salt, or magnesium biotinate is administered alone. In some embodiments, the biotin, biotin salt, or magnesium biotinate is administered in combination with another treatment. In some embodiments, the biotin, biotin salt, or magnesium biotinate is administered for 1 or more days. In some embodiments, the biotin, biotin salt, or magnesium biotinate is administered for 1 or more weeks. In some embodiments, the biotin, biotin salt, or magnesium biotinate is administered for 1 or more months. In some embodiments, the biotin, biotin salt, or magnesium biotinate is administered for 1 or more years.

### BRIEF DESCRIPTION OF THE DRAWINGS

FIG. 1 shows the effects of biotin and magnesium biotinate (MgB) supplementation on body weight and various biochemical parameters in lysolecithin (LPC) induced demyelination of rats. LPC: lysolecithin; B1: Biotin 0.9 mg/rat/day; B2: Biotin 9 mg/rat/day; MgB1: Magnesium biotinate (Biotin 0.9 mg/rat/day); MgB2: Magnesium biotinate (Biotin 9 mg/rat/day). T-C: Total cholesterol; ALT: Alanine aminotransferase; AST: Aspartate aminotransferase. Data presented as mean and standard error. Different superscripts (a-c) indicate group mean differences (p<0.05).

FIG. 2 shows the effects of biotin and magnesium biotinate (MgB) supplementation on serum and brain magnesium, biotin and malondialdehyde concentrations in lysolecithin (LPC) induced demyelination of rats. LPC: lysolecithin; B1: Biotin 0.9 mg/rat/day; B2: Biotin 9 mg/rat/day; MgB1: Magnesium biotinate (Biotin 0.9 mg/rat/day); MgB2: Magnesium biotinate (Biotin 9 mg/rat/day). MDA: Malondialdehyde. Data presented as mean and standard error. Different superscripts (a-c) indicate group mean differences (p<0.05).

FIGS. 3A AND 3B show the effects of biotin and magnesium biotinate (MgB) supplementation on entries to target quadrant (FIG. 3A) and probe trial (FIG. 3B) in the Morris water maze task in lysolecithin (LPC) induced demyelination of rats. B1: Biotin 0.9 mg/rat/day; B2: Biotin 9 mg/rat/day; MgB1: Magnesium biotinate (Biotin 0.9 mg/rat/day); MgB2: Magnesium biotinate (Biotin 9 mg/rat/day). Different superscripts (a-c) indicate group mean differences (p<0.05).

FIG. 4 shows the effects of biotin and magnesium biotinate (MgB) supplementation on latency to find the hidden platform from the first day to the fifth day in the Morris water maze task in lysolecithin (LPC) induced demyelination of rats. B1: Biotin 0.9 mg/rat/day; B2: Biotin 9 mg/rat/day; MgB1: Magnesium biotinate (Biotin 0.9 mg/rat/day); MgB2: Magnesium biotinate (Biotin 9 mg/rat/day).

FIGS. 5A-5F show the effects of biotin and magnesium biotinate (MgB) supplementation on brain tumor necrosis factor alpha (FIG. 5A), interleukin 6 (FIG. 5B), interleukin 17 (FIG. 5C), chemokine (C-C motif) ligand 3 (FIG. 5D), chemokine (C-C motif) ligand 5 (FIG. 5E) and chemokine (C-X-C motif) ligand 16 (FIG. 5F) protein levels in lysolecithin (LPC) induced demyelination of rats. Data are expressed as percent of the control value. Each bar represents the mean and standard error of the mean. Blots were repeated at least 3 times Western blot analysis was performed with actin included to ensure equal protein loading. The data are percentages of the control. a-d: Values within the bars with different superscripts are significantly different (Turkey's post-hoc test, P<0.05). B1: Biotin 0.9 mg/rat/day; B2: Biotin 9 mg/rat/day; MgB1: Magnesium biotinate (Biotin 0.9 mg/rat/day); MgB2: Magnesium biotinate (Biotin 9 mg/rat/day).

FIGS. 6A-6C show the effects of biotin and magnesium biotinate (MgB) supplementation on brain nuclear factor kappa light chain enhancer of activated B cells (FIG. 6A), osteoprotegerin (FIG. 6B) and matrix metallopeptidase 9 (FIG. 6C) protein levels in lysolecithin (LPC) induced demyelination of rats. Data are expressed as percent of the control value. Each bar represents the mean and standard error of the mean. Blots were repeated at least 3 times Western blot analysis was performed with actin included to ensure equal protein loading. The data are percentages of the control. a-d: Values within the bars with different superscripts are significantly different (Turkey's post-hoc test, P<0.05). B1: Biotin 0.9 mg/rat/day; B2: Biotin 9 mg/rat/day; MgB1: Magnesium biotinate (Biotin 0.9 mg/rat/day); MgB2: Magnesium biotinate (Biotin 9 mg/rat/day).

FIGS. 7A-7E show the effects of biotin and magnesium biotinate (MgB) supplementation on brain acetyl CoA carboxylase 1 (FIG. 7A), acetyl CoA carboxylase 2 (FIG. 7B), pyruvate carboxylase (FIG. 7C), propionyl-CoA carboxylase (FIG. 7D), 3-methylcrotonyl-CoA carboxylase (FIG. 7E) protein levels in lysolecithin (LPC) induced demyelination of rats. Data are expressed as percent of the control value. Each bar represents the mean and standard error of the mean. Blots were repeated at least 3 times Western blot analysis was performed with actin included to ensure equal protein loading. The data are percentages of the control. a-d: Values within the bars with different superscripts are significantly different (Turkey's post-hoc test, P<0.05). B1: Biotin 0.9 mg/rat/day; B2: Biotin 9 mg/rat/day; MgB1: Magnesium biotinate (Biotin 0.9 mg/rat/day); MgB2: Magnesium biotinate (Biotin 9 mg/rat/day).

FIGS. 8A-8D show the effects of biotin and magnesium biotinate (MgB) supplementation on brain-derived neurotrophic factor (FIG. 8A), growth-associated protein (FIG. 8B), glial fibrillary acidic protein (FIG. 8C) and intercellular adhesion molecule 1 (FIG. 8D) protein levels in lysolecithin (LPC) induced demyelination of rats. Data are expressed as percent of the control value. Each bar represents the mean and standard error of the mean. Blots were repeated at least 3 times Western blot analysis was performed with actin included to ensure equal protein loading. The data are percentages of the control. a-d: Values within the bars with different superscripts are significantly different (Turkey's post-hoc test, P<0.05). B1: Biotin 0.9 mg/rat/day; B2: Biotin 9 mg/rat/day; MgB1: Magnesium biotinate (Biotin 0.9 mg/rat/day); MgB2: Magnesium biotinate (Biotin 9 mg/rat/day).

FIGS. 9A-9C show the effects of biotin and magnesium biotinate (MgB) supplementation on brain Synapsin-I (FIG. 9A), postsynaptic density protein 93 (FIG. 9B) and postsynaptic density protein 95 (FIG. 9C) protein levels in lysolecithin (LPC) induced demyelination of rats. Data are expressed as percent of the control value. Each bar represents the mean and standard error of the mean. Blots were repeated at least 3 times Western blot analysis was performed with actin included to ensure equal protein loading. The data are percentages of the control. a-d: Values within the bars with different superscripts are significantly different (Turkey's post-hoc test, P<0.05). B1: Biotin 0.9 mg/rat/day; B2: Biotin 9 mg/rat/day; MgB1: Magnesium biotinate (Biotin 0.9 mg/rat/day); MgB2: Magnesium biotinate (Biotin 9 mg/rat/day).

### DETAILED DESCRIPTION

The terminology used in the description presented herein is not intended to be interpreted in any limited or restrictive manner, simply because it is being utilized in conjunction with a detailed description of certain specific embodiments described herein. Furthermore, embodiments described herein can include several novel features, no single one of which is solely responsible for its desirable attributes or which is essential to practicing the embodiments described herein.

As used herein, “treat,” “treatment,” or “treating,” refers to administering or providing a composition for prophylactic and/or therapeutic purposes.

As used herein, the terms “prophylactic treatment,” “prevent,” or “preventing,” can refer to treating a subject who does not yet exhibit symptoms of a disease or condition, but who is susceptible to, or otherwise at risk of, a particular disease or condition, whereby the treatment reduces the likelihood that the subject will develop the disease or condition. A “disorder” is any condition that would benefit from treatment with the compositions described herein.

The term “biotin” means D-biotin, an essential water-soluble vitamin also known as Vitamin H, Coenzyme R, or vitamin B7. D-Biotin has Chemical Abstracts Service Registry No. 58-85-5 and the general formula:

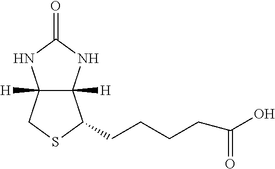

As used herein, the term “biotin salt” refers to an organic, or inorganic salt of D-biotin. Typical salts include alkali metal, alkaline earth metal, ammonia, or organic amine salts as, for example, sodium, potassium, magnesium, calcium, protonated amines such as those derived from ethylamine, triethylamine, ethanolamine, diethylamino-ethanol, ethylenediamine, piperidine, morpholine, 2-piperidinoethanol, benzylamine, procaine and the like

As used herein, the term “magnesium biotinate” refers to the magnesium salt of D-biotin, including magnesium hemi-biotinate. Magnesium D-biotinate is the magnesium salt of the carboxylic acid D-biotin, and does not occur naturally. In some embodiments, magnesium D-biotinate is a stable, non-hygroscopic, off-white powder having a defined composition, a molecular formula of Mg(C₁₀H₁₅N₂O₃S)₂ and a general formula of

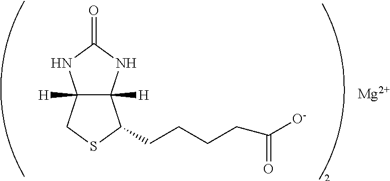

Some embodiments provide physiologically compatible magnesium biotinate hydrates, crystalline forms, polymorphic forms, solid forms having specific bulk densities or tap densities, and solid forms having specific particle sizes. Some embodiments provide compositions coated with pharmaceutically acceptable materials intended to modify its release and/or bioavailability, including, but not limited to Eudragit, microcrystalline cellulose, hydroxypropylmethylcellulose phthalate, and the like.

As used herein, the term “magnesium” refers to the magnesium ion, Mg²⁺.

As used herein, the term “pharmaceutically acceptable solvent” can refer to water, water for injection, aqueous buffer solutions that are physiologically compatible, or aqueous solutions containing organic solvents that are physiologically compatible. A non-comprehensive list of pharmaceutically acceptable solvents is provided in U.S. Department of Health & Human Services, Food & Drug Administration, “Guidance for Industry: Q3C Impurities: Residual Solvents,” December 1997 or its current issue.

As used herein, the term “bioavailability” refers to the amount of a substance that is absorbed in the intestines and ultimately available for biological activity in a subject's tissue and cells.

As used herein, the term “excipient material” refers to any compound that is part of a formulation that is not an active ingredient, i.e., one that has no relevant biological activity, and which is added to the formulation to provide specific characteristics to the dosage form, including by way of example, providing protection to the active ingredient from chemical degradation, facilitating release of a tablet or caplet from equipment in which it is formed, and so forth.

For oral administration, the compositions disclosed herein can be provided as a tablet, aqueous or oil suspension, dispersible powder or granule, emulsion, hard or soft capsule, syrup, elixir, or beverage. Solid dosage forms such as tablets and capsules may comprise an enteric coating. Compositions intended for oral use can be prepared according to any method known in the art for the manufacture of pharmaceutically acceptable compositions and such compositions may include one or more of the following agents: sweeteners, flavoring agents, coloring agents, coatings, and preservatives. The sweetening and flavoring agents will increase the palatability of the preparation. Tablets containing the complexes in admixture with non-toxic pharmaceutically acceptable excipients suitable for tablet manufacture are acceptable. Pharmaceutically acceptable vehicles such as excipients are compatible with the other ingredients of the formulation (as well as non-injurious to the patient). Such excipients include inert diluents such as calcium carbonate, sodium carbonate, lactose, calcium phosphate or sodium phosphate; granulating and disintegrating agents, such as corn starch or alginic acid; binding agents such as starch, gelatin or acacia; and lubricating agents such as magnesium stearate, stearic acid or talc. Tablets can be uncoated or can be coated by known techniques to delay disintegration and absorption in the gastrointestinal tract and thereby provide a sustained action over a longer period of time. For example, a time delay material such as glyceryl monostearate or glyceryl distearate alone or with a wax can be employed.

Formulations for oral use can also be presented as hard gelatin-containing or non-gelatinous capsules wherein the biotin, biotin salt, or magnesium biotinate is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil. Aqueous suspensions can contain the complex of the biotin, biotin salt, or magnesium biotinate admixed with excipients suitable for the manufacture of aqueous suspensions. Such excipients include suspending agents, dispersing or wetting agents, one or more preservatives, one or more coloring agents, one or more flavoring agents and one or more sweetening agents such as sucrose or saccharin.

Oil suspensions can be formulated by suspending the biotin, biotin salt, or magnesium biotinate in a vegetable oil, such as arachis oil, olive oil, sesame oil or coconut oil, or in a mineral oil such as liquid paraffin. The oil suspension can contain a thickening agent, such as beeswax, hard paraffin or cetyl alcohol. Sweetening agents, such as those set forth above, and flavoring agents can be added to provide a palatable oral preparation. These compositions can be preserved by an added antioxidant such as ascorbic acid. Dispersible powders and granules suitable for preparation of an aqueous suspension by the addition of water can provide the biotin, biotin salt, or magnesium biotinate in admixture with a dispersing or wetting agent, a suspending agent, and one or more preservatives. Additional excipients, for example sweetening, flavoring and coloring agents, can also be present.

Syrups and elixirs can be formulated with sweetening agents, such as glycerol, sorbitol or sucrose. Such formulations can also contain a demulcent, a preservative, a flavoring or a coloring agent.

Compositions for parenteral administration can be in the form of a sterile injectable preparation, such as a sterile injectable aqueous or oleaginous suspension. This suspension can be formulated according to methods well known in the art using suitable dispersing or wetting agents and suspending agents. The sterile injectable preparation can also be a sterile injectable solution or suspension in a non-toxic parenterally-acceptable diluent or solvent, such as a solution in 1,3-butanediol. Suitable diluents include, for example, water, Ringer's solution and isotonic sodium chloride solution. In addition, sterile fixed oils can be employed conventionally as a solvent or suspending medium. For this purpose, any bland fixed oil can be employed including synthetic mono or diglycerides. In addition, fatty acids such as oleic acid can likewise be used in the preparation of injectable preparations.

Aqueous suspensions may contain the biotin, biotin salt, or magnesium biotinate in admixture with excipients suitable for the manufacture of aqueous suspensions. Such excipients include suspending agents, dispersing or wetting agents, one or more preservatives, one or more coloring agents, one or more flavoring agents and one or more sweetening agents such as sucrose or saccharin.

Controlled release vehicles are well known to those of skill in the pharmaceutical sciences, and these aspects can be applied to nutritional and dietary supplements. The technology and products in this art are variably referred to as controlled release, sustained release, prolonged action, depot, repository, delayed action, retarded release and timed release; the words “controlled release” as used herein is intended to incorporate each of the foregoing technologies.

Numerous controlled release vehicles are known, including biodegradable or bioerodable polymers such as polylactic acid, polyglycolic acid, and regenerated collagen. Known controlled release drug delivery devices include creams, lotions, tablets, capsules, gels, microspheres, liposomes, ocular inserts, minipumps, and other infusion devices such as pumps and syringes. Implantable or injectable polymer matrices, and transdermal formulations, from which active ingredients are slowly released, are also well known and can be used in the disclosed methods.

Controlled release preparations can be achieved by the use of polymers to form complexes with or absorb the biotin, biotin salt, or magnesium biotinate. The controlled delivery can be exercised by selecting appropriate macromolecules such as polyesters, polyamino acids, polyvinylpyrrolidone, ethylenevinyl acetate, methylcellulose, carboxymethylcellulose, and protamine sulfate, and the concentration of these macromolecule as well as the methods of incorporation are selected in order to control release of biotin, biotin salt, or magnesium biotinate.

Controlled release of biotin, biotin salt, or magnesium biotinate can be taken to mean any of the extended release dosage forms. The following terms may be considered to be substantially equivalent to controlled release, for the purposes of the present disclosure: continuous release, controlled release, delayed release, depot, gradual release, long term release, programmed release, prolonged release, programmed release, proportionate release, protracted release, repository, retard, slow release, spaced release, sustained release, time coat, time release, delayed action, extended action, layered time action, long acting, prolonged action, sustained action medications and extended release, release in terms of pH level in the gut and intestine, breakdown of the molecule and based on the absorption and bioavailability.

Hydrogels, wherein biotin, biotin salt, or magnesium biotinate is dissolved in an aqueous constituent to gradually release over time, can be prepared by copolymerization of hydrophilic mono-olefinic monomers such as ethylene glycol methacrylate. Matrix devices, wherein biotin, biotin salt, or magnesium biotinate is dispersed in a matrix of carrier material, can be used. The carrier can be porous, non-porous, solid, semi-solid, permeable or impermeable. Alternatively, a device comprising a central reservoir of magnesium biotinate surrounded by a rate controlling membrane can be used to control the release of the complex. Rate controlling membranes include ethylene-vinyl acetate copolymer or butylene terephthalate/polytetramethylene ether terephthalate. Use of silicon rubber or ethylene-vinyl alcohol depots are also contemplated.

Controlled release oral formulations are also well known. In one embodiment, the active complex is incorporated into a soluble or erodible matrix, such as a pill or a lozenge. In another example, the oral formulations can be a liquid used for sublingual administration. These liquid compositions can also be in the form a gel or a paste. Hydrophilic gums, such as hydroxymethylcellulose, are commonly used. A lubricating agent such as magnesium stearate, stearic acid, or calcium stearate can be used to aid in the tableting process.

Biotin, biotin salt, or magnesium biotinate may also be delivery topically, including in a salve, cream, lotion, ointment, shampoo, cosmetic, or emulsion.

The compositions may be administered once, twice, three times per day, or more. In some aspects, the compositions are administered four times a day. For example, the compositions may be administered before, after, or during a meal. Dosing for oral administration may be with a regimen calling for single daily dose, or for a single dose every other day, or for a single dose within 72 hours of the first administered dose, or for multiple, spaced doses throughout the day. In some embodiments, wherein biotin, a biotin salt, or magnesium biotinate is combined with another treatment in a combination therapy, the biotin, biotin salt, or magnesium biotinate and the other active agents which make up the combination therapy may be administered simultaneously, either in a combined dosage form or in separate dosage forms intended for substantially simultaneous oral administration. The biotin, biotin salt, or magnesium biotinate and the other active agents which make up the combination therapy may also be administered sequentially, with either the biotin, biotin salt, or magnesium biotinate and the other active component being administered by a regimen calling for two-step ingestion. Thus, a regimen may call for sequential administration of the biotin, biotin salt, or magnesium biotinate and the other active agents with spaced-apart ingestion of the separate compositions. The time period between the multiple ingestion steps may range from a few minutes to as long as about 72 hours, depending upon the properties of each composition such as potency, solubility, bioavailability, plasma half-life and kinetic profile of the agent, as well as depending upon the age and condition of the patient. The compositions of the combination therapy, i.e., biotin, biotin salt, or magnesium biotinate and the other active agents, whether administered simultaneously, substantially simultaneously, or sequentially, may involve a regimen calling for administration of one composition by oral route and the other composition by intravenous route. Whether the compositions of a combined therapy are administered by oral or intravenous route, separately or together, each such composition will be a suitable pharmaceutical formulation of pharmaceutically-acceptable excipients, diluents or other formulations components.

Active ingredients (e.g., biotin, biotin salt, or magnesium biotinate and the other active ingredients of a combination therapy) can be administered by the oral route in solid dosage forms, such as tablets, capsules, and powders, or in liquid dosage forms, such as elixirs, syrups, and suspensions. The biotin, biotin salt, or magnesium biotinate and the other active ingredients of a combination therapy can be administered by the parenteral route in liquid dosage forms. The composition can be made in the form of a dosage unit containing a particular amount of each active ingredient. One example of an oral dosage form of a composition of the present application is an admixture of powders contained within a sachet. Because a composition of the present application is not hygroscopic and has no repugnant taste or odor, the admixture of powders comprising a composition of the present application can be sprinkled on food or stirred into beverages to enhance ease of use and support high levels of compliance with daily dosage regimens.

In general, the dosage forms of compositions of this disclosure can be prepared by conventional techniques, as are described in Remington's Pharmaceutical Sciences, a standard reference in this field [Gennaro AR, Ed. Remington: The Science and Practice of Pharmacy. 20^{th} Edition. Baltimore: Lippincott, Williams & Williams, 2000]. For therapeutic purposes, the active components of a single, oral combination therapy application can be combined with one or more adjuvants appropriate to the indicated route of administration. The components may be admixed with lactose, sucrose, starch powder, cellulose esters of alkanoic acids, cellulose alkyl esters, talc, stearic acid, magnesium stearate, gelatin, acacia gum, sodium alginate, polyvinylpyrrolidone, and/or polyvinyl alcohol, and then tableted or encapsulated for convenient administration, the amounts of which are ascertainable by the skilled artisan. Such capsules or tablets may contain a controlled-release formulation as may be provided in a dispersion of active compound in hydroxypropyl methylcellulose. Solid dosage forms can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract. Both the solid and liquid oral dosage forms can contain coloring and flavoring to increase patient acceptance. Other adjuvants and modes of administration are well and widely known in the pharmaceutical art and these aspects can also be applied to any of the nutritional or dietary supplements described herein.

While the present invention has been described in some detail for purposes of clarity and understanding, one will appreciate that various changes in form and detail can be made without departing from the true scope of the invention.

### EXAMPLES

### Example 1

Male Wistar rats were purchased from the Firat University Laboratory Animal Research Center (Elazig, Turkey). The animals were reared at the temperature of 22±2° C., humidity of 55±5% and with a 12 h light-12 h dark cycle. All animal procedures were approved by the Animal Experimentation Ethics Committee of Firat University (Elazig, Turkey). Rats were divided into 6 groups:

1. Control group were administered 1.5 μl of sterile saline bilaterally into the CA1 region of hippocampi as a LPC solvent control.

2. LPC group, were administered 1.5 μl of LPC 1% bilaterally into the CA1 region of hippocampi and 2 μl of sterile saline intraventricular (i.c.v) as drugs solvent 5 days after LPC injection up to the end of experiment.

3. Biotin1: were administered LPC into the CA1 region of hippocampi and five days later d-biotin was administered daily at low dose (equivalent to a 30 mg human dose) 0.9 mg/rat/day (via gavage) for 4 weeks.

4. Biotin2: were administered LPC into the CA1 region of hippocampi and five days later d-biotin was administered daily at high dose (equivalent to a 300 mg human dose) 9 mg/rat/day (via gavage) for 4 weeks.

5. MgB1: were administered LPC into the CA1 region of hippocampi and five days later magnesium biotinate was administered daily at low dose (equivalent to a 30 mg human dose) 0.9 mg/rat/day (via gavage) for 4 weeks.

6. MgB2: were administered LPC into the CA1 region of hippocampi and five days later magnesium biotinate was administered daily at high dose (equivalent to a 300 mg human dose) 9 mg/rat/day (via gavage) for 4 weeks.

Elemental biotin amount was calculated based on 30 or 300 mg biotin that is needed for a 70-kg adult human after adjusting doses based on metabolic body size (70^{0.75}=24.20 kg, needing 100 mg; ˜0.250^{0.75}=0.71 kg needing 2.95 mg (2954.4 μg=˜3 mg). At the end of the study, blood samples were collected by cardiac puncture after an overnight fast and all rats were sacrificed by cervical dislocation. The brain samples were removed after sacrificing the animals.

Demyelination was induced by stereotaxic injection of lysolecithin (LPC) as previously described (Pourabdolhossein et al., 2014). Briefly, animals were anesthetized with ketamine (100 mg/kg, Alfasan, Holland) and xylazine (10 mg/kg, Alfasan, Holland) and were positioned in a stereotaxic device in a skull flat situation. A single dose of 1.5 μl of LPC 1% (Sigma, St. Louis, Mo., USA) in 0.9% saline was injected bilaterally into the CA1 region of hippocampi, using appropriate stereotaxic coordinates (AP=−3.6 from bregma; ML=±1.6; DV=−3.2 from dura surface). The control group received an equal volume of sterile saline into the CA1 area. The needle was kept in place for an additional 3 min to equilibrate tissue and inject solution to avoid the possible reflux through the needle tract.

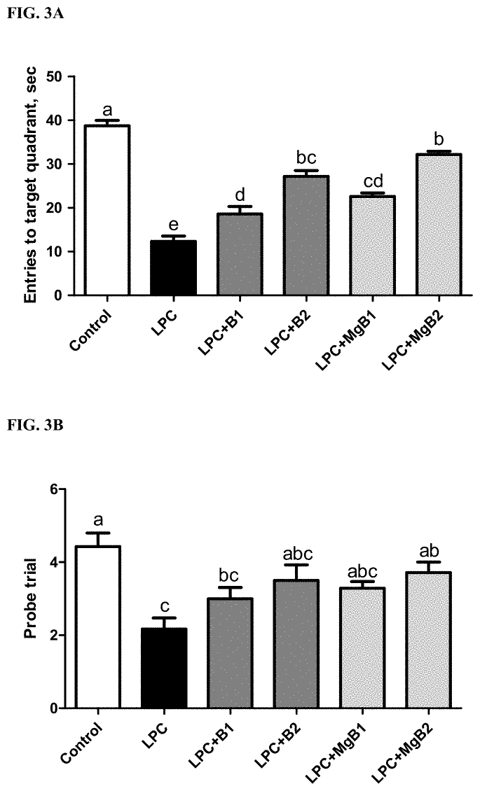

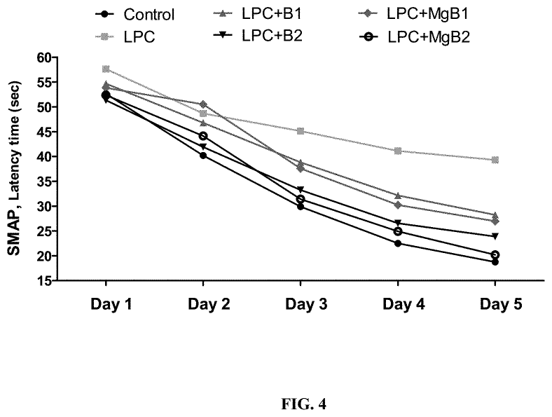

The water maze test was performed as previously described (Wang et al., 2014). The water maze was conducted in large circular black pool (160 cm in diameter) containing water (temperature at 24±2° C.) that had been colored with a nontoxic black dye to contrast the rat. A 12-cm-diameter black-colored round platform was placed 1.5 cm below the water surface. All of the rats were placed in the water maze room 1 h before the water maze trial daily. The rats were given a maximum time of 60 s to find the hidden platform, and they were allowed to remain on the platform for 30 s. The rats were guided to land on the platform if they failed to find the platform within 60 s. The rats were given a daily session of four trials per day for six consecutive days. The swimming pathway and latency in locating the hidden platform were recorded for each trial. On the seventh day, the number of crossings and the percent time spent in the target quadrant were tested with the platform removed. Results are presented in FIGS. 3A-4.

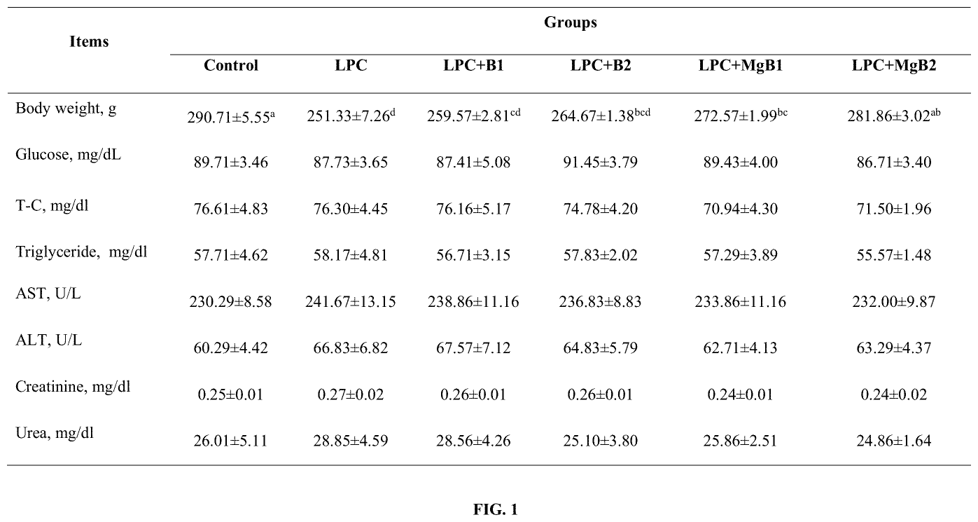

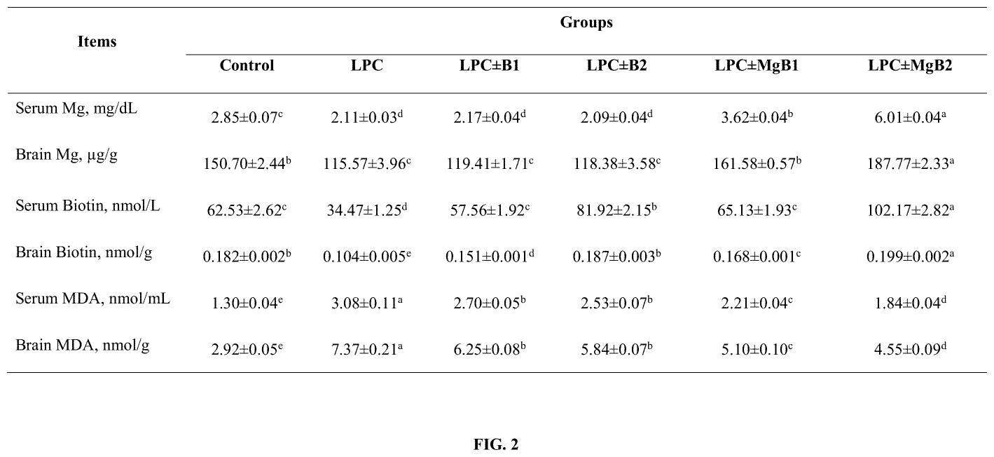

Sera samples were prepared by centrifuging the blood at 3.000×g for 10 min and used for the analyses of biochemical parameters. Serum parameters were determined using an automated analyzer (Samsung LABGEOPT10, Samsung Electronics Co, Suwon, Korea). The concentration of serum and brain biotin and Mg were measured. The measurement of biotin in sera and brain were performed with a HPLC as previously described with minor modifications. Results are presented in FIGS. 1 and 2.

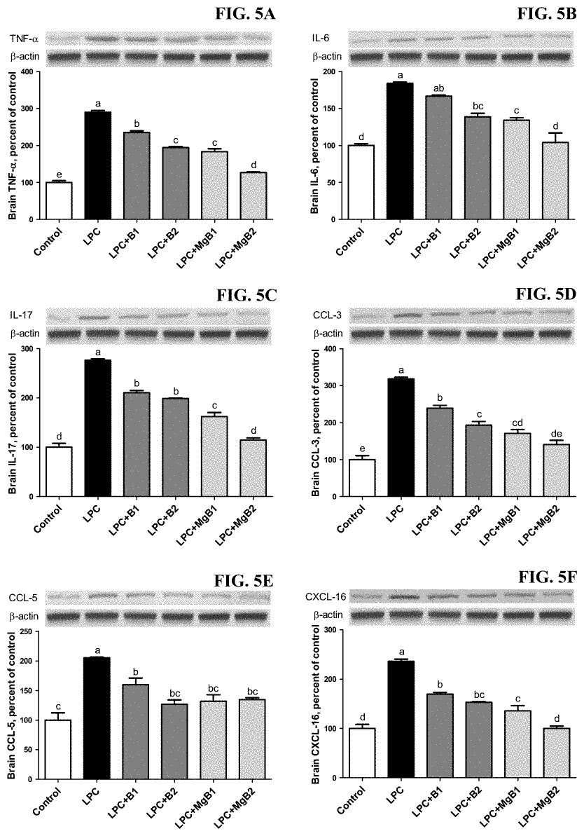

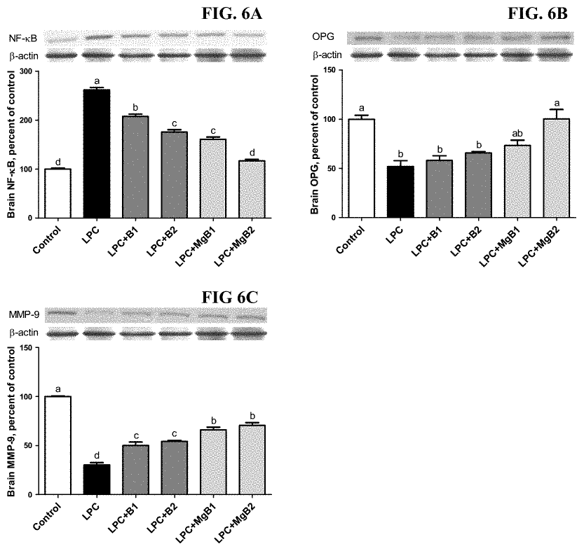

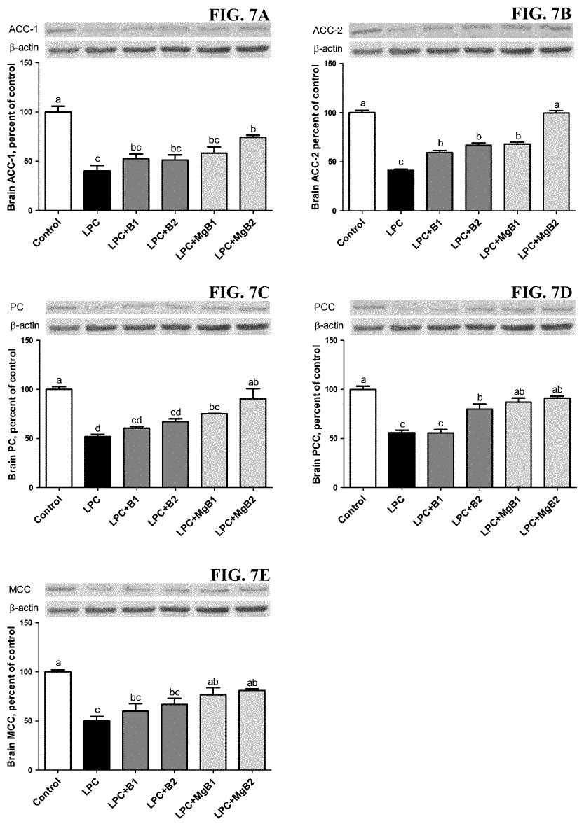

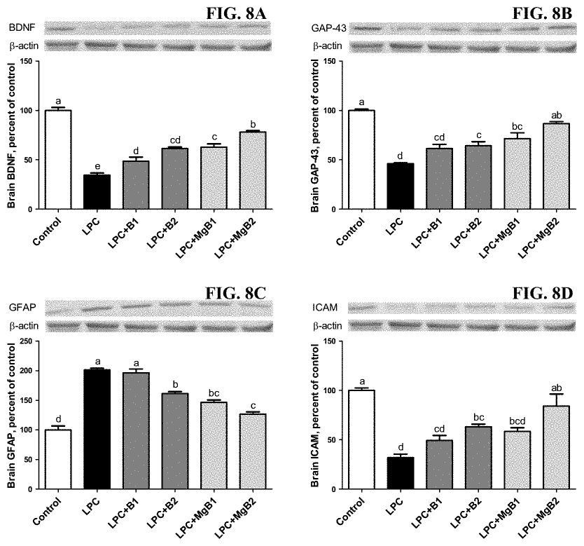

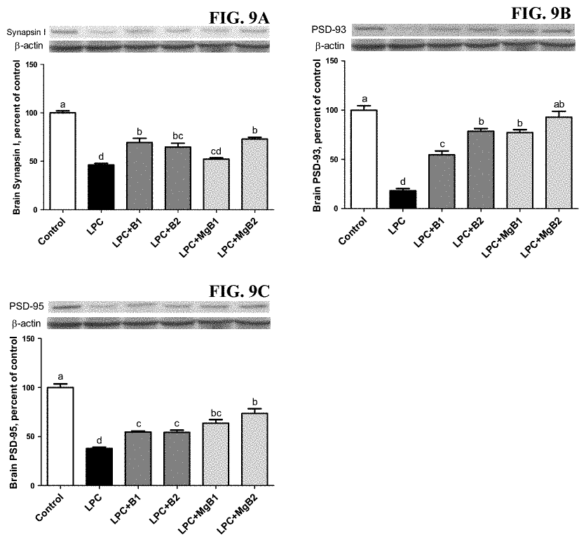

Western blotting was performed to determine brain levels of the following compounds AC, ACC1, ACC2, PC, PCC, presynaptic synapsin I, postsynaptic PSD95, PSD93, IL-17, IL-6, TNF-α, NFκB, GFAP, GAP43, ICAM-1, BDNF, CXCL 16, OPG and MMP-9 levels. Statistical analysis using one-way analysis of variance (ANOVA) followed by the Tukey post hoc test (SAS Institute: SAS User's Guide: Statistics), and P<0.05 was performed. Data were stated as mean±SE. Results are presented in FIGS. 5A-9C.

What is claimed is:

1. A method of treating autoimmune disorders of a human central nervous system comprising administering an effective amount of biotin.

2. The method of claim 1, wherein the autoimmune disorder of a human's central nervous system is selected from the group consisting of clinical initial syndrome (CIS) and relapsing remitting multiple sclerosis (RRMS).

3. The method of claim 2, wherein the autoimmune disorder of a human's central nervous system is CIS.

4. The method of claim 2, wherein the autoimmune disorder of a human's central nervous system is RRMS.

5. The method of claim 1, wherein the autoimmune disorder of a human's central nervous system comprises elevated levels of pro-inflammatory cytokines.

6. The method of claim 1, wherein the effective amount of biotin is between about 10 mg and about 1000 mg.

7. The method of claim 1, wherein the biotin is administered one or more times a day for one or more days.

8. The method of claim 1, wherein the biotin is administered via an oral route, an intraperitoneal route, a transdermal route, a rectal route, or a sublingual route.

9. The method of claim 1, wherein the biotin can be provided as a drug, supplement, medical food, food or biologic.

10. The method of claim 1, wherein the biotin is administered alone.

11. The method of claim 1, wherein the biotin is administered in combination with another treatment.

12. The method of claim 1, wherein the biotin is administered for 1 or more weeks.

13. The method of claim 1, wherein the biotin is administered for 1 or more months.

14. The method of claim 1, wherein the biotin is administered for 1 or more years.

15. A method of delaying progression from CIS to RRMS comprising administering an effective amount of biotin.

16. A method of delaying progression from RRMS to PPMS comprising administering an effective amount of biotin.

17. A method of reducing a level of one or more pro-inflammatory cytokines in a human comprising administering an effective amount of biotin, wherein the level of one or more pro-inflammatory cytokines is reduced.

18. The method of claim 17, wherein the one or more pro-inflammatory cytokines are selected from the group consisting of tumor necrosis factor alpha (TNF-α), interleukin 6 (IL-6), interleukin 17 (IL-17), chemokine (C-C motif) ligand 3 (CCL-3), chemokine (C-C motif) ligand 5 (CCL-5) and chemokine (C-X-C motif) ligand 16 (CXCL-16).
